(19) United States
(12) Patent Application Publication
(10) Pub. No.: US 2004/0211086 A1
(43) Pub. Date: Oct. 28, 2004

(54) Device for high-heeled shoes

(75) Inventor: Howard J. Dananberg, Bedford, NH (US)

Correspondence Address: HAYES, SOLOWAY P.C., 130 W. CUSHING STREET, TUCSON, AZ, 85701, US

(73) Assignee: HBN SHOE, LLC

(21) Appl. No.: 10/421,403

(22) Filed: Apr. 23, 2003

(51) Int. Cl. A43B 3/10; A43B 3/12; A43B 13/38; A43B 23/00; A61F 5/14

(52) U.S. Cl. 36/11.5; 36/7.5; 36/43; 36/174

(57) ABSTRACT

A device for insertion in a high-heeled shoe having a toe box and a heel, has a first raised area in a region underlying the forward edge of a wearer's heel bone, and a second raised area underlying the metatarsals of the wearer.

The present invention relates, in general, to footwear, and more particularly to a footwear construction for walking and other active use. The invention has particular utility in connection with high-heeled shoes, and will be particularly described in connection with such utility, although other utilities are contemplated.

Conventional high-heeled shoes have a reputation of being extremely uncomfortable. There is survey information indicating that as many as 20% of the users of such shoes experience foot pain related to the shoes immediately, and the majority of users experience such pain after as little as four hours of use.

With reference to FIG. 1 there is shown in ghost the bone structure of a foot in a conventional high-heeled shoe. The foot structure comprises the heel bones 10 (also known as tarsus), including the talus and calcaneous, the arch including the cuneiform 11 and the metatarsus 12, and the toe bones or phalanges 13. The heel bones 10 are supported in the shoe by the heel seat 19, while the arch extends over a shank-reinforced midsole region 15 of the shoe to the ball of the foot 16 where the metatarsals join the base of the phalanges 13. The ball of the foot underlying the heads of the metatarsals, forming the metatarsus, and the phalanges are supported by the toe region 9 of the shoe.

The shoe illustrated in FIG. 1 has a heel height of approximately 2 inches (approximately 5 cm). The particular shoe illustrated is based on a U.S. standard ladies' shoe size 7. The shoe is shown on a ground plane 18 from which the heel height “a” to the heel seat 19 is measured. The heel seat 19 is inclined upwardly at an angle of approximately 12-15 degrees relative to the ground plane. This angle is referenced “X” in FIGS. 1 and 3. The shank-reinforced midsole region 15 is angled downwardly and forwardly of the shoe from the heel seat at an angle of approximately 30 degrees relative to the ground plane. This angle is referenced “Y” in FIGS. 1 and 3. In the toe region of the shoe the great toe is essentially parallel to the ground plane, but may be inclined upwardly and forwardly by the upward and forward inclination of the toe region at an angle of 2-3 degrees relatively to the ground plane 18. This angle is referenced “Z” in FIGS. 1 and 2.

As will be appreciated, a conventional high-heeled shoe such as shown in FIG. 1 places the wearer's foot essentially on an inclined plane, whereupon the foot is urged forward by gravity into the toe box in standing or walking. This results in pressure on the ball or forefoot regions and toe jamming which often gives rise to a burning sensation in these areas of the foot, as well as fatigue and discomfort.

In my prior PCT/US97/17656 published Application, I describe a shoe construction having a heel seat having a slightly downwardly-inclined angle (relative to the shank plane), and an arch support extending forward from the heel seat. The shoe includes a rigid shaped device 19 comprising a heel seat 20 and an anatomically shaped arch support 22 running distally from the heel to just proximal the metatarsal heads, affixed as a structural element of the shoe. The rigid shaped device includes a deep conformal heel cup, and runs distally from the heel of the shoe to the point just behind the metatarsal head area, and is molded and/or machined to conform to the underside of the foot whereby to cause the foot to assume an anatomically correct position within the shoe.

The combination of the heel seat 20, and the contour of the arch support 22 as an extension of the heel seat, prevents forward sliding of the foot of the wearer in the shoe, and results in higher percentage of the weight of the wearer being carried by and supported by the heel seat than in the case of a conventional high-heeled shoe. Pressure measurements taken within the shoe under the wearer's heel seat and balls of the foot, respectively, of a high-heeled shoe made in accordance with my aforesaid published PCT Application, and a conventional high-heeled shoe of equal heel height, show a shoe made in accordance with my aforesaid published PCT Application provides an increase in the weight carried by the wearer's heel of at least 20%, with a corresponding decrease in the weight carried by the ball of the foot of the wearer, during walking. Thus, as a consequence of a design of the shoe of my aforesaid published PCT Application, a greater portion of the weight of the wearer is borne by the heel seat rather than being transferred by the sloping shank of the shoe to the ball of the foot, toe jamming is prevented, and comfort assured.

While the invention described in my aforesaid published PCT Application provides significant improvements in comfort without affecting the aesthetics of high-heeled shoes and has been adopted by several shoe companies and shoe component manufacturers, there is a need for a low-cost alternative to the rigid device of my aforesaid PCT Application, which may be readily manufactured and inserted into a conventional shoe manufacturing process at little added cost.

### SUMMARY OF THE INVENTION

In accordance with the present invention, there is provided a device for mounting over or under the insole or sock liner of a conventional high-heeled shoe, which device serves to locate and stabilize the heel of the wearer in the shoe whereby the heel of the wearer is retained in a near parallel relationship to the ground plane, and causes the foot to assume an anatomically correct position in the shoe whereby to buttress the phalanges of the wearer to prevent forward sliding of the foot of the wearer relative to the shoe. A feature and advantage of the device of the present invention is that the device may be universally applied to conventional high-heeled shoes without the need to otherwise modify the shoes, or the shoe last. The device according to the present invention may be incorporated into the manufacturing process, or applied as the last step in the manufacturing process, e.g., by fixing the device in position on the insole top surface of the finished shoe. The device also may be applied post-manufacture, i.e., as an after-market device.

### BRIEF DESCRIPTION OF THE DRAWINGS

Further features and advantages of the present invention will be seen from the following detailed description, taken in conjunction with the accompanying drawings, wherein:

FIG. 1 is a diagrammatic cross-sectional view of a conventional high-heeled shoe made in accordance with the prior art;

FIG. 2 is a diagrammatic cross-sectional view of a high-heeled shoe made in accordance with my aforesaid published PCT Application PCT/US97/17656;

FIG. 3 is a diagrammatic, partially-exploded view of the device of the present invention, in a conventional high-heeled shoe, with the skeletal outline of a human foot shown disposed thereon;

FIG. 4 is a top plan view of the device of FIG. 3;

FIG. 5 is a side cross-sectional view of the device of FIG. 4, taken along plane “I-I”;

FIG. 6 is a side cross-sectional view of the device of FIG. 4, taken along plane “II-II;” and

FIGS. 7 and 8 are views, similar to FIG. 3, of alternative embodiments of the invention.

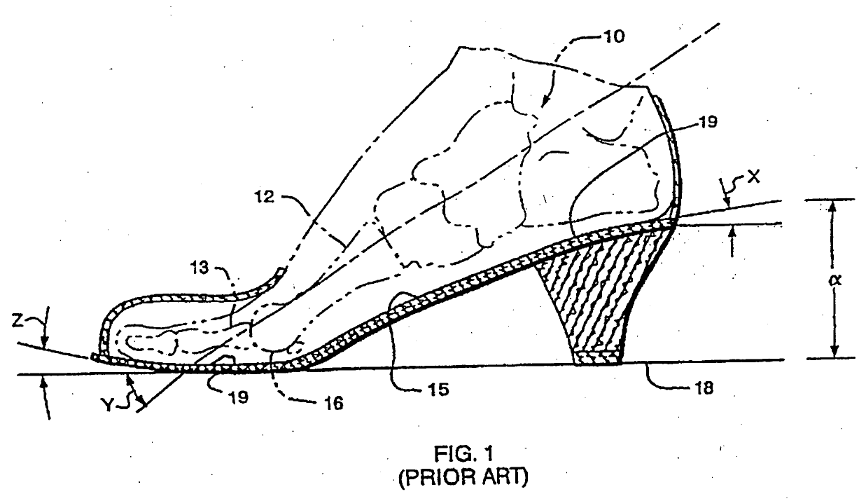

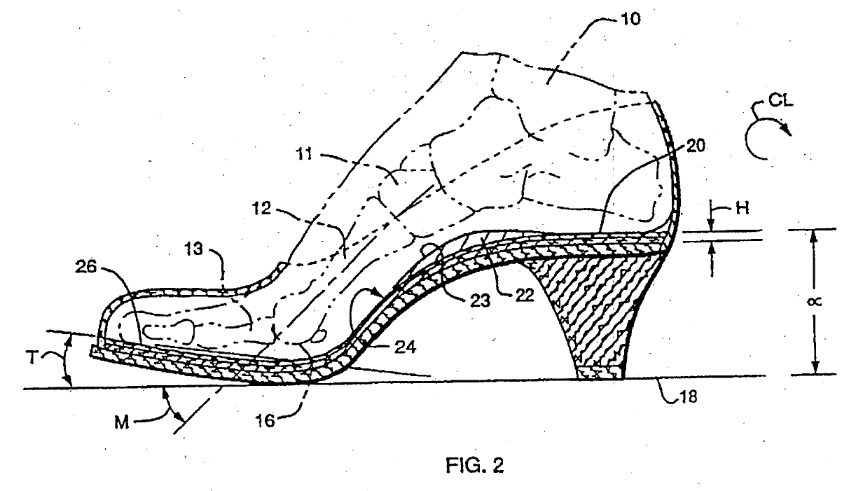

### DETAILED DESCRIPTION OF PREFERRED EMBODIMENT

In the present invention, a device is provided for installation in high-heeled shoes. The device comprises a semi-rigid or flexible shaped device formed of a plastic or rubber-like material, and adhered to the midsole, insole board or sock liner of a high-heeled shoe. The device may be formed of any of the several semi-rigid or flexible materials known to those of ordinary skill in the art, that can be molded or shaped. The device has two distinct raised areas, a first distinct raised area comprising a tapered crescent-shaped rise of about 3-5 millimeters, preferably 3.5-4.5 millimeters at its high point, and located within a shoe to underlie the forward edge of the superior surface of the calceneum of the wearer's foot, and a second distinct raised area, of rounded or tear-drop shape having a high point of about 4-7 millimeters, preferably about 6 millimeters, located within a shoe to underlie the metatarsal shafts of the wearer's foot. The first and second raised areas are joined by a bridge or central area of reduced thickness underlying the arch area of the wearer's foot, i.e., the midsole area of the shoe.

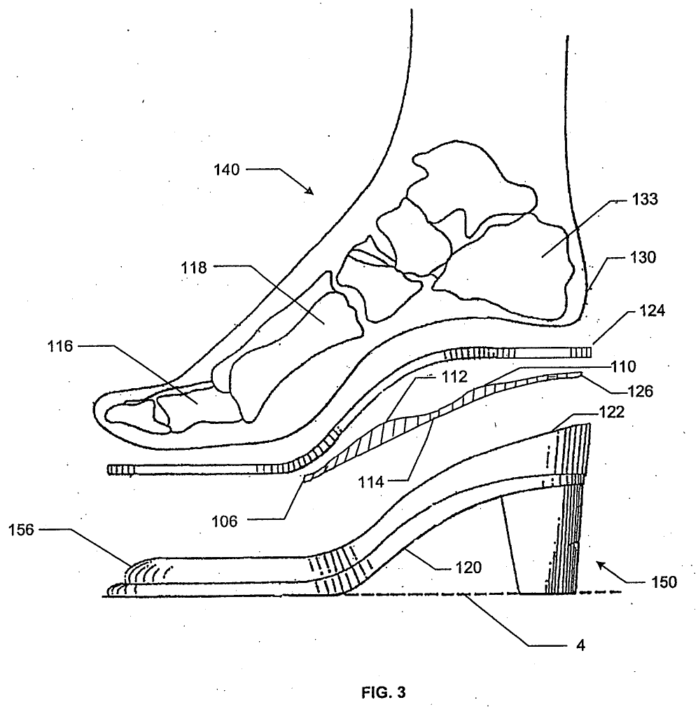

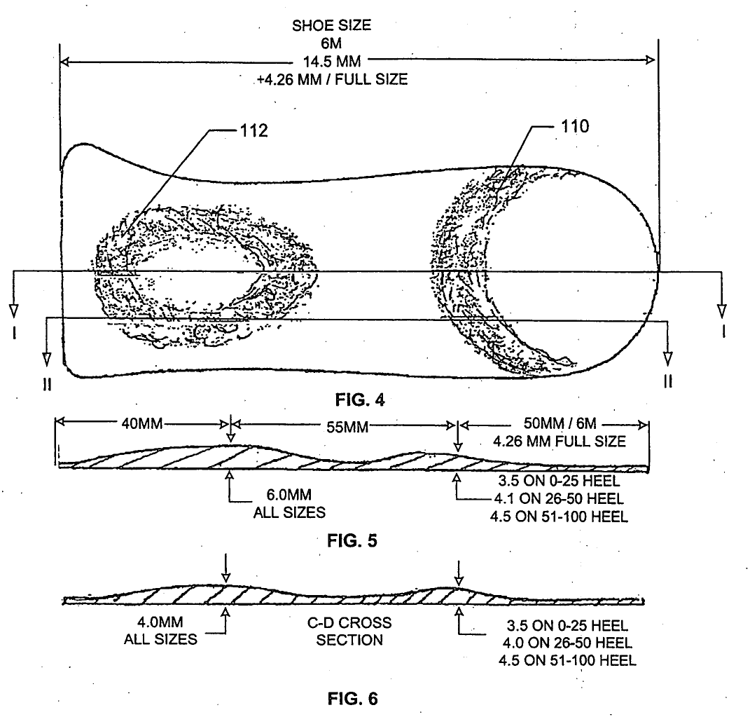

Referring to FIGS. 3-6, which illustrate an exemplary embodiment of a device 102 consistent with the invention. The device 102 is formed from a flexible or semi-flexible material, e.g., molded plastic or rubber, such as polyurethane, TPR, PVC or EVA. The device 102 comprises a metatarsal end 106 and a heel end 126. The device includes two raised areas 110 and 112. The first raised area 110 is generally crescent-shaped and positioned in a shoe to underlie substantially immediately in front of the anterior edge of the heel bone 133 or calceneum of the wearer's foot. The first raised area 110 has a rounded contour to avoid discomfort from sharp edges, and the distal end of the first raised area 110 tapers quickly to a bridge area which connects the first raised area 110 with the second raised area 112. As will be described below in Tables I-III, raised area 110 should have a height, at its apex, i.e., at cross-section I, and a height at cross-section II, so that the heel 130 of the wearer's foot 140 will be positioned so that it rests on a surface that is substantially parallel to or inclined backward to the ground plane 4.

The second raised area 112 is placed forward of the first raised area, to underlie the metatarsal shafts 118 of the wearer's foot. The second raised area comprises a generally rounded shape, preferably in the form of a segmented teardrop, and preferably has a thinner aspect located towards the heel end 126 and a wider aspect located towards the front end 106. The second raised area 112 also has a rounded contour, and should have a height, at its apex, i.e., at cross-section I, and a height at cross-section II, as described below in Tables I-III.

TABLE I
up to 25 mm heel height

| | First Raised | | Second Raised | | | |
|---|---|---|---|---|---|---|
| U.S. Shoe | Area 110 | | Area 112 | | | Heel Cup |
| Size | I | II | I | II | Span⁽¹⁾ | Size⁽²⁾ |
| 5/6 | 3.5 mm | 3.5 mm | 6.0 mm | 4.0 mm | 55 mm | 50 mm |
| 7/8 | 3.5 mm | 3.5 mm | 6.0 mm | 4.0 mm | 59.5 mm | 54 mm |
| 9/10 | 3.5 mm | 3.5 mm | 6.0 mm | 4.0 mm | 64 mm | 58 mm |
| 11/12 | 3.5 mm | 3.5 mm | 6.0 mm | 4.0 mm | 68.5 mm | 62 mm |

⁽¹⁾Distance between the apex of first raised area (110) and second raised area (112). The span is approximately 55 mm for a size 6 shoe, plus 2.25 mm/full size.
⁽²⁾Distance from heel end (126) to apex of first raised area (110). The heel cup size is approximately 50 mm for a size 6 shoe, plus 2 mm/full size.

TABLE II
26-50 mm heel height

| | First Raised | | Second Raised | | | |
|---|---|---|---|---|---|---|
| U.S. Shoe | Area 110 | | Area 112 | | | Heel Cup |
| Size | I | II | I | II | Span⁽¹⁾ | Size⁽²⁾ |
| 5/6 | 4.0 mm | 4.0 mm | 6.0 mm | 4.0 mm | 55 mm | 50 mm |
| 7/8 | 4.0 mm | 4.0 mm | 6.0 mm | 4.0 mm | 59.5 mm | 54 mm |
| 9/10 | 4.0 mm | 4.0 mm | 6.0 mm | 4.0 mm | 64 mm | 58 mm |
| 11/12 | 4.0 mm | 4.0 mm | 6.0 mm | 4.0 mm | 68.5 mm | 62 mm |

⁽¹⁾Distance between the apex of first raised area (110) and second raised area (112). The span is approximately 55 mm for a size 6 shoe, plus 2.25 mm/full size.
⁽²⁾Distance from heel end (126) to apex of first raised area (110). The heel cup size is approximately 50 mm for a size 6 shoe, plus 2 mm/full size.

TABLE III
51-100 mm heel height

| | First Raised | | Second Raised | | | |
|---|---|---|---|---|---|---|
| U.S. Shoe | Area 110 | | Area 112 | | | Heel Cup |
| Size | I | II | I | II | Span⁽¹⁾ | Size⁽²⁾ |
| 5/6 | 4.5 mm | 4.5 mm | 6.0 mm | 4.0 mm | 55 mm | 50 mm |
| 7/8 | 4.5 mm | 4.5 mm | 6.0 mm | 4.0 mm | 59.5 mm | 54 mm |
| 9/10 | 4.5 mm | 4.5 mm | 6.0 mm | 4.0 mm | 64 mm | 58 mm |
| 11/12 | 4.5 mm | 4.5 mm | 6.0 mm | 4.0 mm | 68.5 mm | 62 mm |

⁽¹⁾Distance between the apex of first raised area (110) and second raised area (112). The span is approximately 55 mm for a size 6 shoe, plus 2.25 mm/full size.
⁽²⁾Distance from heel end (126) to apex of first raised area (110). The heel cup size is approximately 50 mm for a size 6 shoe, plus 2 mm/full size.

The bridging section or area of the device between the first raised area 110 and second raised area 112, and the distal ends of the device, i.e., underlying the back of the heel and forward of raised area 112 are relatively thin, typically 1-2 millimeters.

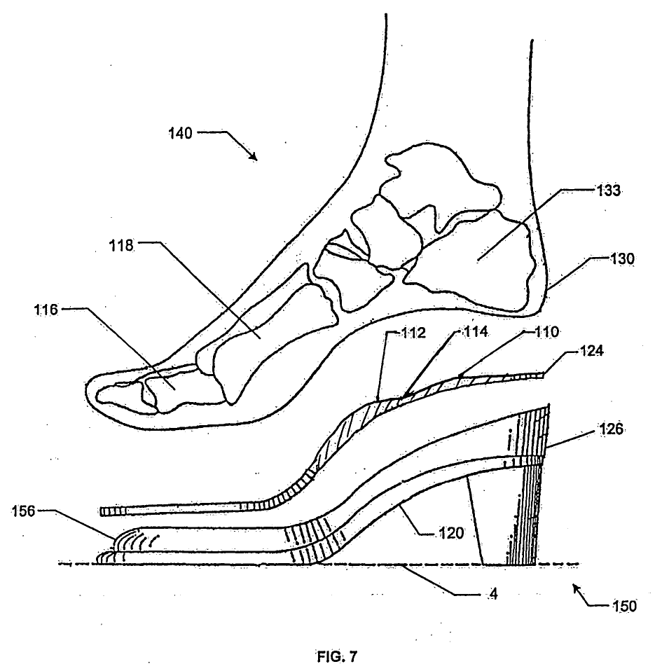

The device 102 preferably is attached to the shoe 150 during the manufacturing process, although it is contemplated that the device 102 may be installed post-manufacture or post-sale in certain embodiments, e.g., by adhesion to the insole or sock liner 124 post-manufacture. In a preferred embodiment of the invention, the device 102 may be glued to the midsole board 122 of the shoe 150, and then a sock liner 124 or insole adhered to the top of the device 102. Alternatively, the device 102 may be attached to the midsole board 122 and the sock liner 124 through means, other than glue, such as a pressure-sensitive adhesive (PSA), hook and loop, e.g., Velcro®, or mechanical fasteners such as nails, staples, etc., to locate and fix the device in the shoe. Device 102 also may be formed integrally with the sock liner, midsole or insole, e.g., as shown in FIG. 7, i.e., by molding.

In use, the device 102 is located within a high-heeled shoe 150 with its heel end 126 at the back of the shoe so that the first raised area 110 underlies substantially immediately in front of the anterior edge of the heel bone of the wearer. From there, the device 102 extends forwardly to a bridging area 114 bridging the first 110 and second 112 raised areas. The second raised area 112 is located to underlie the joints formed by the metatarsal shafts 118 and the phalanges 116. The second raised area 112 tapers toward the distal end 106 of the device 102. Thus, when a foot 140 is inserted into a shoe 150 that houses the device 102, the first raised area 110 serves to locate the heel 130 on a substantially planar surface that is substantially parallel to and/or slightly rearwardly inclined to the ground plane 4. The second raised area 112 positions and stabilizes and supports the metatarsal shafts 118 of the foot 140 so that the metatarsal shafts are shaped and cradled over the second raised area. It should be noted that contrary to the teachings of the prior art, rather than providing a raised area for supporting the arch of the wearer's foot in the device of the present invention, the area underlying the arch is lowered. That is to say, in the shoe midsole area where a traditional arch support normally would be located, in the present invention, the area underlying the arch of the foot is hollowed, leaving the arch unsupported. Unexpectedly, the combination of a hollow under the arch and raised areas immediately in front of the anterior edge of the heel bone and under the metatarsals improves the comfort of shoes made in accordance with the present invention.

The device of the present invention provides many advantages over the prior art. For one, the heel 130 of the wearer's foot 140 is located and carried by the device 102 such that the heel is substantially parallel to or inclined slightly backwardly to the ground plane 4. This greatly reduces the forward inclination force of the wearer's foot 140 toward the toe part 156 of the shoe 150 and therefore prevents the toes 34 of the foot 140 from becoming jammed therein, thus increasing general foot comfort and also improving posture. Thus, the device eliminates the toe pain and general lower back pain associated with the wearing of heeled shoes, particularly high-heeled shoes. Also, since the metatarsal shafts 118 are positioned and supported over the second raised area 112, the metatarsals and phalanges 116 are free to evert and plantarflex under load in a proper biomechanical motion. Thus, foot pain, endemic with the use of high-heeled shoes, is reduced or eliminated using the instant device.

The instant invention permits many variations without departing from the instant invention. For example, the thickness and thickness distribution of the first raised area 110 may be adapted as necessary in order to maintain the heel 130 of the wearer's foot 140 in substantially parallel or slightly backwardly inclined relationship to the ground plane 4. Thus, for example, in heels having 1″ or less height, the thickness of the first raised area 110 will be less, while for shoes with greater heel height, for instance, 2″, the thickness of the first raised area 110 may be made greater so as to accommodate the increased angle of the midsole 120 of the shoe 150 relative to the ground plane 4. Similarly, the size of the device 102, the distance between the first 110 and second 112 raised areas, and other dimensions may vary based upon the size of the shoe and heel height. Also, the sizes given above in TABLES I-III are based on U.S. women's shoe sizes. To convert to English, European or Japanese shoe sizes, the following TABLE IV should be consulted.

TABLE IV
WOMEN'S SHOE SIZES

| | | | | | | | |
|---|---|---|---|---|---|---|---|
| U.S.A. | 3.5 | 4.0 | 4.5 | 5.0 | 5.5 | 6.0 | 6.5 |
| English | 2.0 | 2.5 | 3.0 | 3.5 | 4.0 | 4.5 | 5.0 |
| European | 34.0 | 35.0 | 35.5 | 36.0 | 37.0 | 37.5 | 38.0 |
| Japanese | 21.5 | 22.0 | 22.5 | 23.0 | 23.5 | 24.0 | 24.5 |
| U.S.A. | 7.0 | 7.5 | 8.0 | 8.5 | 9.0 | 9.5 | 10.0 |
| English | 5.5 | 6.0 | 6.5 | 7.0 | 7.5 | 8.0 | 8.5 |
| European | 39.0 | 39.5 | 40.0 | 40.5 | 41.0 | 42.0 | 42.5 |
| Japanese | 25.0 | 25.5 | 26.0 | 26.5 | 27.0 | 27.5 | 28.0 |

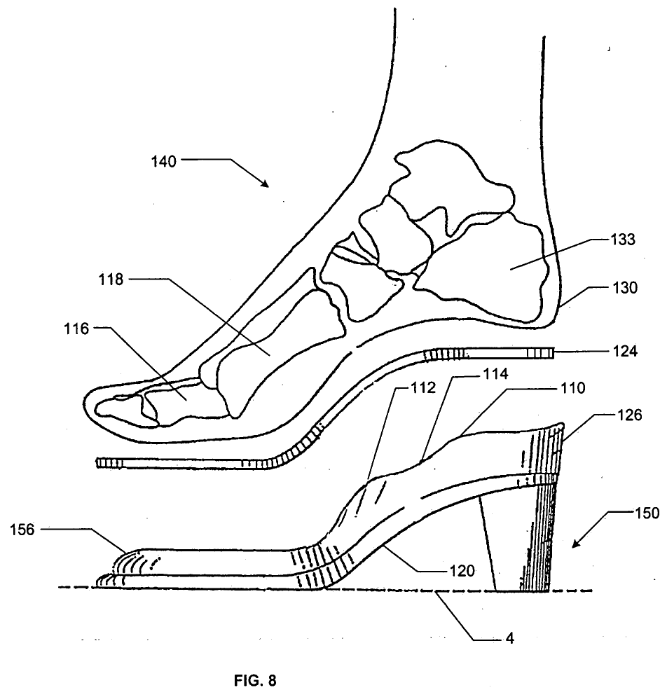

It is, therefore, evident that there is provided, in accordance with the present invention, a device that fully satisfies both the aims and objectives hereinbefore set forth. While this invention has been described in conjunction with specific embodiments thereof, it will be evident to those skilled in the art that many alternatives, modifications, and variations are possible without departing from the scope of the instant invention. For example, as shown in FIG. 8, the insole board or footbed of the shoe could be integrally shaped or molded to conform to the shape of the device. Also, the two raised areas may be made as separate pieces, and individually positioned in a shoe. However, for ease of manufacture, a single device having the separate raised areas joined by a bridging section is preferred. Accordingly, it is intended to embrace all such alternatives, modifications, and variations as fall within the spirit and broad scope of the appended claims.

What is claimed is:

1. A device for insertion in a high-heeled shoe having a toe box and a heel, said device having a first raised area in a region underlying the forward edge of a wearer's heel bone, and a second raised area underlying the metatarsals of the wearer.

2. The device as claimed in claim 1, wherein the first raised area comprises a crescent-shaped raised area that underlies in front of a forward arc of a superior surface of the wearer's calceneum.

3. The device as claimed in claim 2, wherein the crescent-shaped raised area is 3 to 5 mm high at its apex.

4. The device as claimed in claim 1, wherein said second raised area comprises a teardrop-shaped raised area.

5. The device as claimed in claim 4, wherein said teardrop-shaped area has a thinner aspect located toward the heel, and a wider aspect located toward the toe box.

6. The device as claimed in claim 4, wherein the teardrop-shaped raised area is 4 to 7 mm high at its apex.

7. The device as claimed in claim 1, wherein the first raised area at its apex is located about 50 mm from the back of the heel, for a U.S. size 6 shoe, plus about 2 mm per full size.

8. The device as claimed in claim 1, wherein the first raised area and the second raised area are spaced from one another by a distance, measured at their respective apexes, of about mm, for a U.S. size 6 shoe, plus about 2.25 mm per full size.

9. The device as claimed in claim 1, affixed to an insole or sock liner of the shoe.

10. The device as claimed in claim 1, affixed to a footbed of the shoe.

11. The device as claimed in claim 1, affixed to a midsole board of the shoe.

12. The device as claimed in claim 1, formed integrally with a shoe insole, sock liner or midsole.

13. An insole for insertion in a high-heeled shoe having a toe box and a heel, said insole having a first raised area in a region underlying the forward edge of a wearer's heel bone, and a second raised area underlying the metatarsals of the wearer.

14. An insole as claimed in claim 13, wherein the first raised area comprises a crescent-shaped raised area that underlies in front of a forward arc of a superior surface of the wearer's calceneum.

15. An insole as claimed in claim 14, wherein the crescent-shaped raised area is 3 to 5 mm high at its apex.

16. An insole as claimed in claim 13, wherein said second raised area comprises a teardrop-shaped raised area.

17. An insole as claimed in claim 16, wherein said teardrop-shaped area has a thinner aspect located toward the heel, and a wider aspect located toward the toe box.

18. An insole as claimed in claim 16, wherein the teardrop-shaped raised area is 4 to 7 mm high at its apex.

19. An insole as claimed in claim 13, wherein the first raised area at its apex is located about 50 mm from the back of the heel, for a U.S. size 6 shoe, plus about 2 mm per full size.

20. An insole as claimed in claim 13, wherein the first raised area and the second raised area are spaced from one another by a distance, measured at their respective apexes, of about 55 mm, for a U.S. size 6 shoe, plus about 2.25 mm per full size.

21. A sock liner for insertion in a high-heeled shoe having a toe box and a heel, said sock liner having a first raised area in a region underlying the forward edge of a wearer's heel bone, and a second raised area underlying the metatarsals of the wearer.

22. A sock liner as claimed in claim 21, wherein the first raised area comprises a crescent-shaped raised area that underlies in front of a forward arc of a superior surface of the wearer's calceneum.

23. A sock liner as claimed in claim 22, wherein the crescent-shaped raised area is 3 to 5 mm high at its apex.

24. A sock liner as claimed in claim 21, wherein said second raised area comprises a teardrop-shaped raised area.

25. A sock liner as claimed in claim 24, wherein said teardrop-shaped area has a thinner aspect located toward the heel, and a wider aspect located toward the toe box.

26. A sock liner as claimed in claim 24, wherein the teardrop-shaped raised area is 4 to 7 mm high at its apex.

27. A sock liner as claimed in claim 21, wherein the first raised area at its apex is located about 50 mm from the back of the heel, for a U.S. size 6 shoe, plus about 2 mm per full size.

28. A sock liner as claimed in claim 21, wherein the first raised area and the second raised area are spaced from one another by a distance, measured at their respective apexes, of about 55 mm, for a U.S. size 6 shoe, plus about 2.25 mm per full size.

29. A midsole for incorporation into a high-heeled shoe having a toe box and a heel, said midsole having a first raised area in a region underlying the forward edge of a wearer's heel bone, and a second raised area underlying the metatarsals of the wearer.

30. A midsole as claimed in claim 29, wherein the first raised area comprises a crescent-shaped raised area that underlies in front of a forward arc of a superior surface of the wearer's calceneum.

31. A midsole as claimed in claim 30, wherein the crescent-shaped raised area is 3 to 5 mm high at its apex.

32. A midsole as claimed in claim 29, wherein said second raised area comprises a teardrop-shaped raised area.

33. A midsole as claimed in claim 32, wherein said teardrop-shaped area has a thinner aspect located toward the heel, and a wider aspect located toward the toe box.

34. A midsole as claimed in claim 32, wherein the teardrop-shaped raised area is 4 to 7 mm high at its apex.

35. A midsole as claimed in claim 29, wherein the first raised area at its apex is located about 50 mm from the back of the heel, for a U.S. size 6 shoe, plus about 2 mm per full size.

36. A midsole as claimed in claim 29, wherein the first raised area and the second raised area are spaced from one another by a distance, measured at their respective apexes, of about 55 mm, for a U.S. size 6 shoe, plus about 2.25 mm per full size.

37. A high-heeled shoe having a footbed underlying a toe box and a heel, said footbed having a first raised area in a region underlying the forward edge of a wearer's heel bone, and a second raised area underlying the metatarsals of the wearer.

38. A high-heeled shoe as claimed in claim 37, wherein the first raised area comprises a crescent-shaped raised area that underlies in front of a forward arc of a superior surface of the wearer's calceneum.

39. A high-heeled shoe as claimed in claim 38, wherein the crescent-shaped raised area is 3 to 5 mm high at its apex.

40. A high-heeled shoe as claimed in claim 37, wherein said second raised area comprises a teardrop-shaped raised area.

41. A high-heeled shoe as claimed in claim 40, wherein said teardrop-shaped area has a thinner aspect located toward the heel, and a wider aspect located toward the toe box.

42. A high-heeled shoe as claimed in claim 40, wherein the teardrop-shaped raised area is 4 to 7 mm high at its apex.

43. A high-heeled shoe as claimed in claim 37, wherein the first raised area at its apex is located about 50 mm from the back of the heel, for a U.S. size 6 shoe, plus about 2 mm per full size.

44. A high-heeled shoe as claimed in claim 37, wherein the first raised area and the second raised area are spaced from one another by a distance, measured at their respective apexes, of about 55 nun, for a U.S. size 6 shoe, plus about 2.25 mm per full size.
